(19) United States
(12) Patent Application Publication
(10) Pub. No.: US 2024/0252547 A1
(43) Pub. Date: Aug. 1, 2024

(54) COMPOSITION FOR PREVENTING AND TREATING STROKE, COMPRISING HLA HOMOZYGOUS INDUCED PLURIPOTENT STEM CELL-DERIVED NEURAL PRECURSOR CELLS

(71) Applicants: CHA UNIVERSITY INDUSTRY-ACADEMIC COOPERATION FOUNDATION, Pocheon-si (KR); IPS BIO, INC., Seongnam-si (KR)

(72) Inventor: Jihwan SONG, Seoul (KR)

(73) Assignees: CHA UNIVERSITY INDUSTRY-ACADEMIC COOPERATION FOUNDATION, Pocheon-si (KR); IPS BIO, INC., Seongnam-si (KR)

(21) Appl. No.: 18/564,485

(22) PCT Filed: May 26, 2022
(86) PCT No.: PCT/KR2022/007500

§ 371 Date: Mar. 15, 2024

(30) Foreign Application Priority Data
May 27, 2021 (KR) 10-2021-0068608

(51) Int. Cl. A61K 35/30; A61P 9/10; C12N 5/0797

(52) U.S. Cl. CPC A61K 35/30; A61P 9/10; C12N 5/0623; C12N 2500/32; C12N 2500/38; C12N 2500/98; C12N 2501/115; C12N 2506/45; C12N 2533/52

(57) ABSTRACT

A pharmaceutical composition for preventing or treating a stroke, the pharmaceutical composition comprising, as an active ingredient, neural precursor cells differentiated from HLA homozygous human induced pluripotent stem cells (hiPSC-NPC), wherein, through the use of a homozygous type, immunorejection may be eliminated and stable therapeutic effects for stroke may be exhibited.

### CROSS REFERENCE TO RELATED APPLICATIONS

This application is a National Stage entry under 35 U.S.C. § 371 of PCT/KR2022/007500, filed on May 26, 2022, and claims priority to KR Patent Application No. 10-2021-0068608, filed on May 27, 2021, the entire contents of which are incorporated herein by reference.

### TECHNICAL FIELD

The present invention relates to a composition for preventing and treating stroke, the composition comprising HLA homozygous induced pluripotent stem cell-derived neural precursor cells.

### BACKGROUND ART

Ischemic stroke is the most common form of stroke, accounting for approximately 85% of strokes. A stroke occurs when blood flow to the brain is blocked, causing brain cells to die due to a lack of oxygen or nutrients. There is no effective treatment 6 hours after a stroke occurs.

Until now, it was considered impossible to treat brain cell damage caused by stroke, but therapeutic treatments using various stem cells for ischemic stroke have been recently developed. One type of stem cells, a patient-derived induced pluripotent stem cells, has great promise for use as a therapeutic agent. However, the cost and time required to produce a clinical-grade cell line for each patient is enormous, making it unrealistic, and if the patient is elderly or has a genetic disease, it is difficult to guarantee the quality of the cell line produced. Therefore, there is an urgent need to prepare new alternatives.

### DISCLOSURE

### Technical Problem

One aspect is to provide a pharmaceutical composition for preventing or treating stroke, the pharmaceutical composition comprising neural precursor cells differentiated from human leucocyte antigen (HLA) homozygous human induced pluripotent stem cells (hiPSC-NPCs) as an active ingredient.

Another aspect provides a method of producing neural precursor cells differentiated from HLA homozygous human induced pluripotent stem cells, the method comprising a phase of differentiating the HLA homozygous human induced pluripotent stem cells into the neural precursor cells.

### Technical Solution

One aspect provides a pharmaceutical composition for preventing or treating stroke, the pharmaceutical composition comprising as an active ingredient neural precursor cell differentiated from HLA homozygous human induced pluripotent stem cells (hiPSC-NPCs).

In an example, the neural precursor cells differentiated from HLA homozygous human induced pluripotent stem cells (hiPSC-NPCs), may increase immune compatibility in the brain and increase neural connectivity with existing neural tissues by using neural precursor cells, rather than a fully differentiated neuron, as an active ingredient.

The term “human leukocyte antigen (HLA)” is a gene in major histocompatibility complexes (MHC) that help code for proteins that differentiate between self and non-self. It may refer to an antigen that is a genetically diverse glycoprotein expressed on the surface of a white blood cell (leukocyte) and plays a significant role in disease and immune interacting with complement, the cytotoxic effect of T cells, and cellular humoral immunity. Proteins produced by the human leukocyte antigen are expressed on the surface of body cells in a combination unique to each individual, and the immune system may use these antigens to differentiate self or non-self cells.

The number of alleles for the human leukocyte antigen may vary depending on the class. HLAs corresponding to MHC class I (HLA-A, HLA-B, and HLA-C), all of which are the HLA class I group, present peptides from inside the cell. HLAs corresponding to MHC class II (HLA-DP, HLA-DQ, and DR) present antigens from outside of the cell to T-lymphocytes.

The term “homozygote” may refer to a subject with the same allele for one gene. As an opposite concept, a subject with an opposite allele for one gene may be referred to as a heterozygote.

Induced pluripotent stem cells produced by donating somatic cells from a healthy donor with the same HLA homozygote do not have mutations that cause genetic diseases and may be supplied to many recipients with compatible HLA.

The human leukocyte antigen homozygous stem cell or a human leukocyte antigen homozygous stem cell-derived neural precursor cells may be reliably administered to HLA type A, type B, and type DRB1 compatible recipients without immune rejection.

In an embodiment, the HLA homozygote may be homozygous for HLA-A, HLA-B, or HLA-DRB1. Specifically, the HLA homozygote may be homozygous for HLA-A, HLA-B, and HLA-DRB1.

In an example, the HLA homozygote may be *33:03 for HLA-A, *44:03 for HLA-B, and *07:01 for HLA-DRB1.

In an embodiment, the human induced pluripotent stem cell may be derived from a cord blood mononuclear cell (CMC) or peripheral blood mononuclear cells (PBMC).

The term “induced pluripotent stem cells” refers to cells induced to have pluripotent differentiation ability through an artificial dedifferentiation process from differentiated cells and is also called induced pluripotent stem cells (IPSCs). The artificial dedifferentiation process may be performed by a virus-mediated or non-viral vector using a retrovirus or lentivirus, by the introduction of a non-virus-mediated dedifferentiation factor using a protein and cell extract, or includes a dedifferentiation process using a stem cell extract, compound, etc. The induced pluripotent stem cell has almost the same properties as an embryonic stem cell, specifically, exhibiting a similar cell shape, have a similar gene and protein expression pattern, have pluripotency in vitro and in vivo, form a teratoma, form chimeric mice when injected into a blastocyst of mice, and is capable of germline transmission of genes. The induced pluripotent stem cell may include an induced pluripotent stem cell of any origin, including human, monkey, pig, horse, bovine, sheep, dog, cat, mouse, rabbit, etc.

In an example, the neural precursor cell may include one or more selected from the group consisting of Sox2, Nestin, Musashi, Tuj1, GABA, DARPP-32, TH, SVP38, and PSD95. In an embodiment, the neural precursor cell may include one or more selected from the group consisting of Sox2, Nestin, and Musashi. Specifically, the neural precursor cell may include Sox2, Nestin, and Musashi.

The term “precursor cell” refers to a parent cell that does not express a differentiation marker, but has a differentiation fate, when a cell corresponding to the progeny is found to express a specific differentiation marker. For example, for a nerve cell (neuron), a neuroblast (neural stem cell) may correspond to a neural precursor cell.

The term “neural precursor cells (NPCs)” refer to cells at a phase before completely acquiring the form and function of nerve cells. Neural precursor cells according to the above aspect may be cells expressing Nestin, Sox2, and Musashi, etc. These are unique genes expressed in neural precursor cells, and their expression detected in cells represents neural precursor cells.

The term “stroke” refers to a rapidly developing partial or total impairment of brain function that persists for a considerable period of time or longer and may refer to a condition in which no cause other than cerebrovascular disease may be found.

In an embodiment, the stroke may be any one or more selected from cerebral infarction (ischemic stroke), cerebral hemorrhage (hemorrhagic stroke), transient ischemic attack, and recurrent stroke. Specifically, the stroke may be an ischemic stroke or a hemorrhagic stroke.

In an example, the stroke may be in a chronic, subacute, or acute stage. Specifically, the stroke may be a subacute stage that does not exceed 3 months after acute cerebrovascular infarction.

The term “prevent” refers to any act of inhibiting a disease or delaying the onset of a disease by administration of a pharmaceutical composition. The term “treatment” refers to any act of ameliorating or beneficially altering the course of a disease by the administration of a pharmaceutical composition.

The pharmaceutical composition may have the effect of promoting cerebral angiogenesis. The cerebral angiogenesis may be the restoration or regeneration of cerebrovascular vessel destroyed by stroke or subacute stroke, or the generation of a new blood vessel. In an embodiment, the prevention or treatment of stroke may be by cerebral angiogenesis.

In an embodiment, the pharmaceutical composition may increase the number of RECA1⁺ blood vessels by 0.1 to 10 times, 0.1 to 5 times, 0.5 to 2 times, 1.1 to 1.5 times, for example, 1.15 to 1.3 times compared to the control group.

The pharmaceutical composition may have the effect of preventing and ameliorating cerebral neuroinflammation. The cerebral neuroinflammation may be caused by a stroke, and may be caused by a subacute stroke. In an embodiment, the prevention or treatment of stroke may be by preventing and ameliorating cerebral neuroinflammation.

The pharmaceutical composition may have the effect of preventing overproduction of glial scar. A glial scar formation, or gliosis, is a reactive cellular process involving astrogliosis that occurs after injury to the central nervous system. In an embodiment, the prevention or treatment of stroke may be by preventing overproduction of intracerebral glial scar.

The pharmaceutical composition may comprise the active ingredient alone or may be provided as a pharmaceutical composition comprising one or more pharmaceutically acceptable carriers, excipients, or diluents.

Specifically, the carriers may be, for example, a colloidal suspension, a powder, a saline, a lipid, a liposome, a microsphere, or a nanospheric particle. They may form a complex with or be associated with a transport method and may be transported in vivo using transport systems known in the art, such as a lipid, liposome, microparticle, gold, nanoparticle, polymer, condensation reactant, polysaccharide, poly amino acid, dendrimer, saponin, adsorption enhancing substance, or fatty acid.

When the pharmaceutical composition is formulated, it may be prepared using commonly used diluents or excipients such as a lubricant, sweetener, flavoring agent, emulsifier, suspending agent, preservative, filler, extender, binder, humectant, disintegrant and surfactant, etc. Solid preparations for oral administration may include a tablet, pill, powder, granule, capsule, etc., and such solid preparations may be prepared by mixing the composition with at least one or more excipient, for example starch, calcium carbonate, sucrose or lactose, and gelatin. In addition to simple excipients, lubricants such as magnesium stearate and talc may also be used. Liquid preparations for oral use may include a suspension, oral liquid medication, emulsion, and syrup, etc., in addition to the commonly used simple diluents such as water and liquid paraffin, various excipients for example a humectant, sweetener, fragrance, and preservative, etc. Preparations for parenteral administration may include a sterile aqueous solution, non-aqueous solution, suspension, emulsion, lyophilized preparation, and suppository. A propyleneglycol, polyethylene glycol, vegetable oil such as olive oil, and an injectable ester such as ethylolate, etc. may be used as a non-aqueous solution and suspension. As a base for the suppository, witepsol, macrogol, tween 61, cacao butter, laurin butter, glycerogelatin, etc. may be used, and when manufacturing it in the form of an eye drop, known diluent or excipient, etc. may be used.

In an embodiment, the pharmaceutical composition may comprise neural precursor cells in an amount of about 0.01×10⁵ cells/2 μl to 100×10⁷ cells/2 μl, 0.1×10⁵ cells/2 μl to 100×10⁷ cells/2 μl, 0.1×10⁵ cells/2 μl to 20×10⁷ cells/2 μl, 0.1×10⁵ cells/2 μl to 10×10⁷ cells/2 μl, 0.1×10⁵ cells/2 μl to 2×10⁷ cells/2 μl, 0.1×10⁵ cells/2 μl to 0.1×10⁷ cells/2 μl, 0.1×10⁵ cells/2 μl to 100×10⁵ cells/2 μl, 1×10⁵ cells/2 μl to 100×10⁵ cells/2 μl, 5×10⁵ cells/2 μl to 100×10⁵ cells/2 μl, 10×10⁵ cells/2 μl to 100×10⁵ cells/2 μl, 0.1×10⁵ cells/2 μl to 10×10⁵ cells/2 μl, 1×10⁵ cells/2 μl to 10×10⁵ cells/2 μl, or 5×10⁵ cells/2 μl to 10×10⁵ cells/2 μl.

In an embodiment, the pharmaceutical composition may comprise fibroblasts in an amount of about 0.01×10⁵ cells/2 μl to 100×10⁷ cells/2 μl, 0.1×10⁵ cells/2 μl to 100×10⁷ cells/2 μl, 0.1×10⁵ cells/2 μl to 20×10⁷ cells/2 μl, 0.1×10⁵ cells/2 μl to 10×10⁷ cells/2 μl, 0.1×10⁵ cells/2 μl to 2×10⁷ cells/2 μl, 0.1×10⁵ cells/2 μl to 0.1×10⁷ cells/2 μl, 0.1×10⁵ cells/2 μl to 100×10⁵ cells/2 μl, 1×10⁵ cells/2 μl to 100×10⁵ cells/2 μl, 5×10⁵ cells/2 μl to 100×10⁵ cells/2 μl, 10×10⁵ cells/2 μl to 100×10⁵ cells/2 μl, 0.1×10⁵ cells/2 μl to 10×10⁵ cells/2 μl, 1×10⁵ cells/2 μl to 10×10⁵ cells/2 μl, or 5×10⁵ cells/2 μl to 10×10⁵ cells/2 μl.

In an embodiment, if the neural precursor cell is hNu⁺ or hNestin⁺, they may be neural precursor cells with high survival ability. Specifically, if the neural precursor cell is hNestin⁺, it may be a neural precursor cell with high survival rate.

In an embodiment, if the neural precursor cell is hMAP2⁺ or NeuN⁺, it may differentiate into a mature neuron. Specifically, when neural precursor cells are hMAP2⁺ or NeuN⁺, they may differentiate into a GABAergic neuron, a DARPP-32⁺ medium spiny neuron, or a TH⁺ dopaminergic neuron.

Another aspect provides a method of producing neural precursor cells differentiated from HLA homozygous human induced pluripotent stem cells, comprising the phase of differentiating HLA homozygous human induced pluripotent stem cells into neural precursor cells.

In an embodiment, the phase of differentiating HLA homozygous human induced pluripotent stem cells may be performed by culturing the human induced pluripotent stem cells in a medium for neural precursor cell differentiation.

The differentiation medium may include bFGF, ROCK inhibitor, antifungal antibiotic, non-essential amino acid (NEAA), sodium pyruvate, D-glucose, L-glutamine, beta-MeOH, and B-27. In an embodiment, a phase of culturing a formed embryoid body in a medium for natural killer cell differentiation may be performed in a culture vessel coated with poly-L-ornithine or laminin.

Poly-L-ornithine coated on the culture vessel may be included in an amount of 0.5 μg to 2 g, 1 μg to 1 g, 10 μg to 500 mg, 10 μg to 100 mg, 10 μg to 10 mg, 10 μg to 1 mg, 10 μg to 500 μg, 10 μg to 200 μg, or 50 μg to 150 μg per 100 μl of a medium for neural precursor cell differentiation.

Laminin coated on the culture vessel may be included in an amount of 0.5 μg to 2 g, 1 μg to 1 g, 1 μg to 500 mg, 1 μg to 100 mg, 1 μg to 10 mg, 1 μg to 1 mg, 1 μg to 100 μg, 1 μg to 50 μg, 1 μg to 20 μg, or 5 μg to 15 μg per 100 μl of medium for neural precursor cell differentiation.

In an embodiment, a phase of culturing stem cells in a medium for neural precursor cell differentiation may be performed for 1 day to 20 days, 1 day to 15 days, specifically 5 days to 13 days, and more specifically 6 days to 12 days.

In an embodiment, the method may further comprise the phase of forming an embryoid body (EB) containing the neural precursor cells.

In an embodiment, embryoid body may be seeded at a concentration of 5 embryoid bodies to 30 embryoid bodies per 2 ml of differentiation medium. Specifically, the embryoid body may be seeded at a concentration of 10 embryoid bodies to 20 embryoid bodies per 2 ml of differentiation medium. More specifically, the embryoid body may be seeded at a concentration of 12 embryoid bodies to 18 embryoid bodies per 2 ml of differentiation medium. More specifically, the embryoid body may be seeded at a concentration of 14 embryoid bodies to 16 embryoid bodies per 2 ml of differentiation medium.

Detailed descriptions of other ingredients, etc. are as described above.

In an aspect, provided is a stroke treatment kit, comprising neural precursor cells differentiated from HLA homozygous human induced pluripotent stem cells (hiPSC-NPCs).

Detailed descriptions of other ingredients, etc. are as described above.

In an aspect, provided is a method of preventing or treating stroke, comprising the phase of determining whether to administrate neural precursor cells differentiated from HLA homozygous human induced pluripotent stem cells (hiPSC-NPCs) to a subject.

The term “subject” refers to any animal, such as a rat, mouse, or livestock, etc., including a human, in which a stroke has occurred or may occur. As a specific example, the subject may be a mammal other than a human.

The term “administration” refers to introducing a certain substance into a subject in an appropriate method. The administration may be performed directly to the subject with any routes, such as, for example, oral, intravenous, intramuscular, transdermal, mucosal, intranasal, intratracheal or subcutaneous administration. The administration may be performed systemically or locally. The local administration site may be determined to the area where a damaged cerebrovascular vessel is present.

In an embodiment, the pharmaceutical composition may be administered orally or parenterally, and when administered parenterally, the injection method may be selected for external application through the skin, intraperitoneal injection, intrarectal injection, subcutaneous injection, intravenous injection, intramuscular injection, or intrathoracic injection. Specifically, the pharmaceutical composition may be administered in the form of application to the endometrium for external use on the skin, or it may also be injected directly into the endometrium in the form of an injection. More specifically, the pharmaceutical composition may be applied to the endometrium of a subject.

The pharmaceutical composition is administered in a pharmaceutically effective amount. As used herein, the term “pharmaceutically effective amount” refers to an amount sufficient to treat a condition with a reasonable benefit-risk ratio applicable to medical treatment, and the effective dose level may be determined by factors including the type and severity of the patient's condition, the activity of the drug, sensitivity to the drug, time of administration, route of administration and elimination rate, duration of treatment, concomitant medications, and other factors well known in the medical fields. The administration may be performed once a day or may be administered several times.

Specifically, the effective amount of the pharmaceutical composition may vary depending on the patient's age, condition, weight, absorption of the active ingredient in the body, inactivation rate, excretion rate, type of disease, concomitant medication, and may increase or decrease depending on the administration route, severity of obesity, gender, weight, age, etc.

The effective amount may be about 0.5 μg to about 2 g, from about 1 μg to about 1 g, from about 10 μg to about 500 mg, from about 100 μg to about 100 mg, or from about 1 mg to about 50 mg per pharmaceutical composition.

The effective amount may be that the pharmaceutical composition includes about 0.1×10⁷ to 100×10⁷, 0.1×10⁷ to 10×10⁷, 1×10⁷ to 10×10⁷, 1×10⁷ to 5×10⁷, or 2×10⁷ to 4×10⁷ HLA homozygous human induced pluripotent stem cells.

For example, the dosage of the pharmaceutical composition may be in the range of about 0.001 mg/kg to about 100 mg/kg, about 0.01 mg/kg to about 10 mg/kg, or about 0.1 mg/kg to about 1 mg/kg for adults.

In an embodiment, the neural precursor cells may have the effect of reducing the number of ED1⁺-Iba1⁺ cells in the host receiving the composition.

Detailed descriptions of other ingredients, etc. are as described above.

Another aspect provides a pharmaceutical composition for preventing and ameliorating cerebral neuroinflammation, comprising as active ingredient neural precursor cells differentiated from HLA homozygous human induced pluripotent stem cells (hiPSC-NPCs). In addition, provided is a method for preventing and ameliorating cerebral neuroinflammation comprising the phase of administrating the composition to a subject.

Another aspect provides a pharmaceutical composition for preventing overproduction of intracerebral glial scar, comprising as active ingredient neural precursor cells differentiated from HLA homozygous human induced pluripotent stem cells (hiPSC-NPCs). Additionally, provided is a method of preventing overproduction of intracerebral glial scar, comprising the phase of administrating the composition to a subject.

In an embodiment, iNOS-expressing microglia/macrophage may exacerbate brain damage during stroke by secreting pro-inflammatory cytokines such as interleukin-1 beta (IL-1β) and tumor necrosis factor alpha (TNF-α).

In an embodiment, CD206 (also known as mannose receptor)-expressing microglia/macrophage may participate in the healing process by inhibiting abnormal inflammation and phagocytosing waste and dead cells in the damaged area after stroke.

Another aspect provides a pharmaceutical composition for promoting cerebral angiogenesis development, comprising as active ingredient neural precursor cells differentiated from HLA homozygous human induced pluripotent stem cells (hiPSC-NPCs). Additionally, provided is a method of promoting cerebral angiogenesis generation, comprising the phase of administrating the composition to a subject.

### Advantageous Effects

In an example, human leukocyte antigen homozygous induced pluripotent stem cell-derived neural precursor cells may stably show excellent therapeutic effect in cerebrovascular vessel related diseases without immune rejection.

### DESCRIPTION OF DRAWINGS

FIG. 1A is a schematic diagram illustrating a differentiation procedure of (A) hiPSC into NPC and a mature neuron. FIG. 1B is a representative immunocytochemical image (scale bar=100 μm). FIG. 1C is a representative immunocytochemical image of a hiPSCs-NPCs-derived mature neuron (scale bar=50 μm).

FIG. 2A shows results of a rotarod test on behavioral changes in a mouse model after transplantation of neural precursor cells according to an example. FIG. 2B shows results of a stepping test on behavioral changes in a mouse model after transplantation of neural precursor cells according to an example. FIG. 2C shows results of a modified neurological severity score (mNSS) test on behavioral changes in a mouse model after the neural precursor cells according to an example. FIG. 2D shows results of a staircase test on behavioral changes in a mouse model after transplantation of neural precursor cells according to an example. FIG. 2E shows results of an apomorphine-induced rotation test on behavioral changes in a mouse model after transplantation of neural precursor cells according to an example. FIG. 2F shows a recovery rate for each behavioral experiment over 12 weeks after cell transplantation. FIG. 2G (left) shows results of cresyl violet staining. FIG. 2G (right) shows results of a quantitative analysis of infarct size in a culture medium control group (Media), fibroblast control group (Fibroblast), and iPSC-NPC group (iPSC-NPC) (n=7 per group).

FIG. 3A is a schematic diagram of tissue analysis in a rat brain according to an example. FIG. 3B is a representative image of hNu DAB immunostaining of transplanted hiPSC-NPCs in MCAo rats at 12 weeks after transplantation according to an example.

FIG. 4A shows a double immunohistochemical (IHC) staining image for hNestin and hMAP2. FIG. 4B (left) shows another image of double IHC for hNu and hNestin in fibroblast control group (Fibroblast) and iPSC-NPC group (iPSC-NPC). FIG. 4B (center) shows results of a quantitative analysis of hNut cells in the iPSC-NPC group (iPSC-NPC).

FIG. 4B (right) shows results of a quantitative analysis of hNu⁺-hNestin⁺ cells in the iPSC-NPC group (iPSC-NPC). FIG. 4C (left) shows double IHC images for hNestin and Ki67 in the iPSC-NPC group (IPSC-NPC). FIG. 4C (right) shows results of quantitative analysis of hNestin⁺-Ki67⁺ cells in the iPSC-NPC group (iPSC-NPC).

FIG. 5A (left) is a representative double IHC image for hNu and hMAP2. FIG. 5A (right) shows results of quantitative analysis of hNu⁺-hMAP2⁺ cells in the iPSC-NPC group (IPSC-NPC). FIG. 5B (left) shows double IHC images for hMito and NeuN. FIG. 5B (right) shows results of quantitative analysis of hMito⁺-NeuN⁺ cells in the iPSC-NPC group (IPSC-NPC). FIG. 5C (left) shows double IHC images for hNu and GABA. FIG. 5C (right) shows results of a quantitative analysis of hNu⁺-GABA⁺ cells in the iPSC-NPC group (iPSC-NPC). FIG. 5D (left) shows double IHC images for hNu and DARPP-32. FIG. 5D (right) shows results of a quantitative analysis of hNu⁺-DARPP-32⁺ cells in the iPSC-NPC group (iPSC-NPC). FIG. 5E (left) shows double IHC images for hNu and TH. FIG. 5E (right) shows results of a quantitative analysis of hNu⁺-TH⁺ cells in the iPSC-NPC group (iPSC-NPC). FIG. 5F (left) shows double IHC images for hNu and GFAP. FIG. 5F (right) shows results of a quantitative analysis of hNu⁺-GFAP⁺ cells in the iPSC-NPC group (iPSC-NPC).

FIG. 6A (left) is a schematic diagram illustrating an injection site of hiPSC-NPC and Fluoro-Gold (FG). FIG. 6A (right) shows results of a quantitative analysis of a proportion of FG⁺-hNu⁺ cells in the striatum. FIG. 6B is an image of co-localization of FG⁺ (green) and hNut (red) double positive cells in the striatum (yellow).

FIG. 7A (left) are double IHC images for ED1 and Iba1. FIG. 7A (right) shows results of a quantitative analysis of ED1⁺-Iba1⁺ cells in a culture medium control group (Media), fibroblast control group (Fibroblast), and iPSC-NPC group (iPSC-NPC). FIG. 7B (left) are double IHC images for ED1 and CD206. FIG. 7B (right) shows results of a quantitative analysis of ED1⁺-CD206⁺ cells in the culture medium control group (Media), fibroblast control group (Fibroblast), and iPSC-NPC group (iPSC-NPC). FIG. 7C (left) is an IHC image for GFAP (green) in the peri-infarct area. FIG. 7C (center) shows results of a quantitative analysis of an average area of GFAP+glial scar in the peri-infarct area of a culture medium control group (Media), fibroblast control group (Fibroblast), and iPSC-NPC group (iPSC-NPC). FIG. 7C (right) shows results of a quantitative analysis of an average thickness of GFAP+glial scar in the peri-infarct area of the culture medium control group (Media), fibroblast control group (Fibroblast), and iPSC-NPC group (iPSC-NPC).

FIG. 8 (left) are double IHC images for ED1 and INOS. FIG. 8 (right) shows results of a quantitative analysis of a proportion of iNOS⁺-ED1⁺ cells in the culture medium control group (Media), fibroblast control group (Fibroblast), and iPSC-NPC group (iPSC-NPC).

FIG. 9A is an image of GFAP immunostaining in the peri-infarct cortex of a MCAo rat. FIG. 9B (left) shows results of a quantitative analysis of GFAP⁺ glial scar area in the culture medium control group (Media), fibroblast control group (Fibroblast), and iPSC-NPC group (iPSC-NPC). FIG. 9B (Right) shows results of a quantitative analysis of an average thickness of GFAP+ glial scars in the culture medium control group (Media), fibroblast control group (Fibroblast), and iPSC-NPC group (iPSC-NPC).

FIG. 10A are double IHC images for DCX and BrdU. FIG. 10B (left) shows results of a quantitative analysis of DCX⁺ cells in the culture medium control group (Media), fibroblast control group (Fibroblast), and iPSC-NPC group (iPSC-NPC). FIG. 10B (center) shows results of a quantitative analysis of BrdU+ cells in the culture medium control group (Media), fibroblast control group (Fibroblast), and iPSC-NPC group (iPSC-NPC). FIG. 10B (right) shows results of a quantitative analysis of DCX⁺-BrdU⁺ cells in the culture medium control group (Media), fibroblast control group (Fibroblast), and iPSC-NPC group (iPSC-NPC).

FIG. 11A is a DAB immunostaining image for RECA-1. FIG. 11B (left) shows results of a quantitative analysis of the number of blood vessels around the infarction in the culture medium control group (Media), fibroblast control group (Fibroblast), and iPSC-NPC group (iPSC-NPC). FIG. 11B (right) shows results of a quantitative analysis of the number of branch point of blood vessels around the infarction in the culture medium control group (Media), fibroblast control group (Fibroblast), and iPSC-NPC group (iPSC-NPC). FIG. 11C are double IHC images for BrdU and PECAM1. FIG. 11D are double IHC images for STEM121 and vWF.

### MODE FOR INVENTION

The present invention will be described in more detail below with reference to examples and experimental examples. However, these examples and experimental examples are intended to illustrate the present disclosure by way of example and the scope of the disclosure but are not limited thereto.

### Experimental Example 1. Preparation of Human Leukocyte Antigen Homozygous Induced Pluripotent Stem Cell-Derived Neural Precursor Cells

A hiPSC cell line was cultured in Stemfit® Basic02 medium (provided by Ajinomoto, Japan) supplemented with 100 ng/ml basic fibroblast growth factor (bFGF) and 10 μmol/L Y27632 (ROCK inhibitor) in a CO₂ incubator for 7 days before treatment with TrypLE solution (GIBCO) for 5 minutes at 37° C.

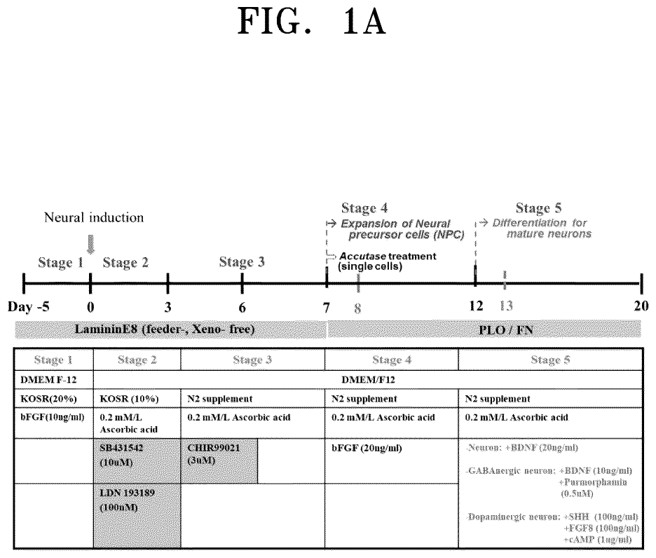

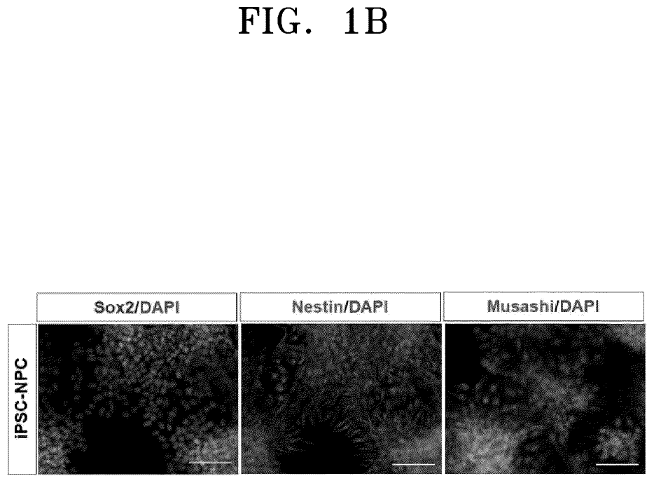

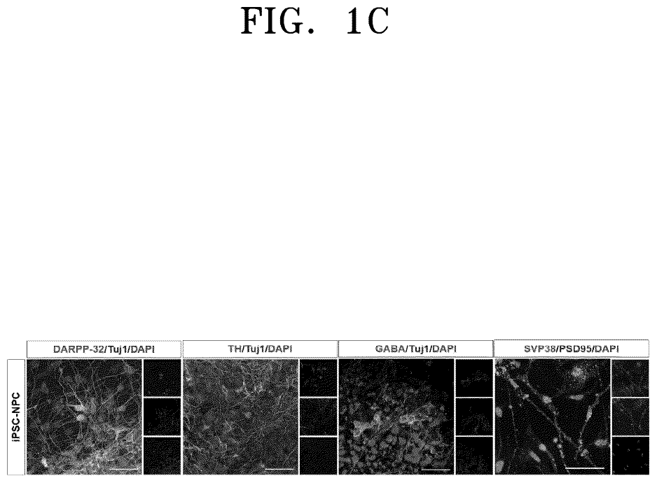

A dissociated hiPSC cell line was incubated in SFEBq medium in a CO₂ incubator at 37° C. for neural induction. A SFEBq medium was consisted of DMEM/F12 (Invitrogen) supplemented with a 1% antifungal antibiotic, 1% non-essential amino acid (NEAA), 0.1% beta-MeOH, 20% Knockout™ SR, 10 μmol/L SB431542, 100 nM LDN193189, and 3× ROCK inhibitor. Cells were maintained in SFEBq medium for 8 days. An embryonic body was dissociated in neural precursor cells medium. The neural precursor cells medium consisted of 1:100 antifungal antibiotics, 1:100 NEAA, sodium pyruvate, D-glucose, L-glutamine, 1:1000 beta-MeOH, 1:50 B-27 (without vitamin A), 20 ng/ml bFGF, and the dish was coated with poly-L-ornithine and laminin (see FIG. 1A). For transplantation, neural precursor cells were isolated with accutase.

### Experimental Example 2. Neural Differentiation and Immunocytochemical Analysis of hiPSC-NPC

### Experimental Example 2.1. Neural Differentiation of hiPSC-NPC

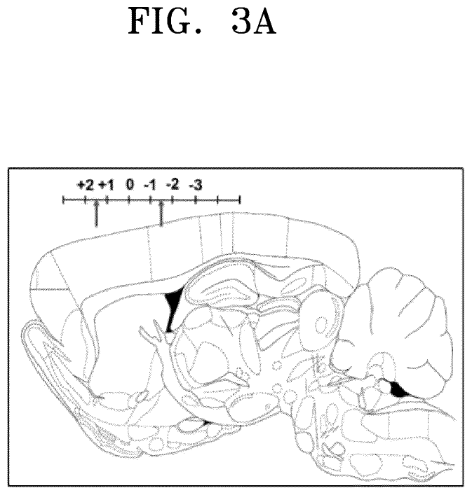

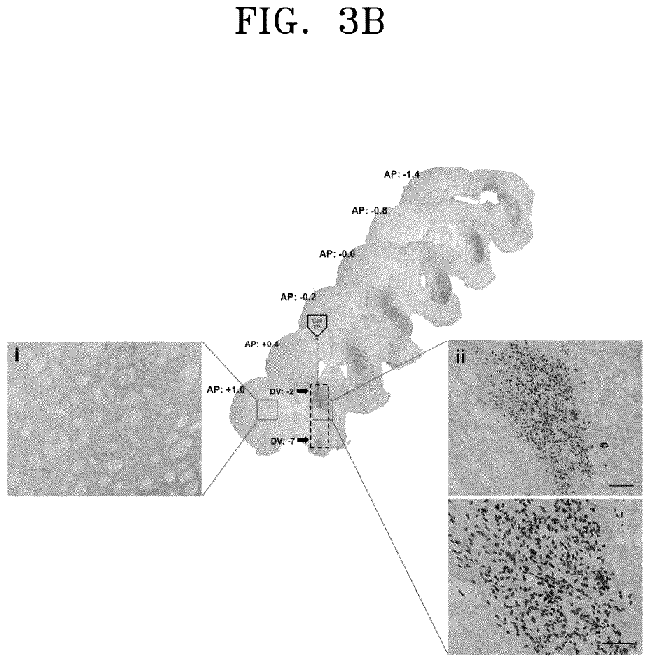

To confirm the differentiation ability of a hiPSC-derived NPC, it was spontaneously differentiated into a mature neuron. In the above process, differentiated hiPSC-NPC was passaged and the medium was changed to mature neuron medium consisting of neurobasal A medium, 1× GlutaMAX, and 1×B27 supplement (see FIG. 3A). For differentiation, 20 ng/ml BDNF for mature neurons, 10 ng/ml BDNF and 0.5 μmol/L permorphamine for GABAergic neurons, and 100 ng/ml SHH, 100 ng/ml FGF8 and 1 μg/mL CAMP for dopaminergic neurons were added to the medium.

The hiPSC was differentiated into a neural precursor cell (NPC) and expressed markers for the NPC such as Sox2, Nestin, and Musashi (see FIG. 1B). Next, the differentiation potential into neurons with the expressions of various neuronal markers such as Tuj1, GABA, TH and DARPP-32 was observed (see FIG. 1C). Tuj1⁺, GABA⁺ and TH⁺ cells were observed at 3 weeks after neural induction, while DARPP-32⁺ cells were observed at 13 weeks after neural induction. Additionally, it was confirmed that neurons derived from hiPSC-NPC showed immunoreactivity for SVP38 and PSD95 at 13 weeks after induction of neural differentiation. It was confirmed that synapse was achieved as the maturation of neurons (see FIG. 1C).

### Experimental Example 2.2. Immunocytochemical Analysis of hiPSC-NPC

Morphological analysis and immunocytochemical staining using antibodies against NPCs and mature neurons during neural differentiation were performed. Cells were fixed with 4% paraformaldehyde for 15 minutes, blocked with 5% normal horse serum/PBS for 30 minutes to prevent non-specific binding after washing with 0.1% Triton X-100/PBS three times. Specific primary antibodies were incubated for 12 hours at 4° C. and then washed three times with PBS (see Table 1), followed by incubation with secondary antibodies.

TABLE 1

| Primary | | Host | | Catalogue |
|---|---|---|---|---|
| antibodies | Dilution | species | Suppliers | number |
| Sox2 | 1:200 | rabbit | Millipore | AB5603 |
| Nestin | 1:200 | rabbit | R&D System | 841901 |
| Musashi | 1:200 | rabbit | Millipore | AB5977 |
| Tuj1 | 1:500 | mouse | Millipore | MAB1637 |
| GABA | 1:500 | rabbit | Sigma-Aldrich | A2052 |
| DARPP-32 | 1:200 | rabbit | Cell Signaling | MBS9407621 |
| TH | 1:200 | rabbit | Pel-Freez | P40101-150 |
| SVP38 | 1:200 | mouse | Abcam | ab8049 |
| PSD95 | 1:200 | rabbit | Cell Signaling | 2507s |

Nuclei were counterstained with 4,6-diamidino-2-phenylindole (DAPI, 1:1000; Roche). Goat anti-mouse IgG Alexa 555 (1:250; Thermo Fisher Scientific), goat anti-rabbit IgG Alexa 488 (1:250; Thermo Fisher Scientific), and goat anti-mouse IgM Alexa 555 (1:250; Thermo Fisher Scientific) were applied as secondary antibodies.

### Experimental Example 3. Preparation of Middle Cerebral Artery Occlusion Rat Model

All animal experiments were conducted in accordance with the Institutional Animal Care and Use Committee (IACUC) guidelines (IACUC150066) of CHA University of Medical Sciences. The stroke model was induced by transient middle cerebral artery occlusion (MCAo) for 90 minutes. An adult male sprague-dawley rat (Seoul Orient, Korea) ranging from 270 g to 300 g was used. After anesthetizing with intraperitoneal (i.p.) injection of 1% ketamine (57.6 mg/kg) and xylazine (7.7 mg/kg), the rat was placed on a heating pad to maintain body temperature at 37±1° C. The right common carotid artery (CCA), external carotid artery (ECA), and internal carotid artery (ICA) were exposed, and a blunt-tipped silicone-coated monofilament (Ethicon, Pinewood, UK; 4-0) was inserted to occlude the middle cerebral artery (MCA) for 90 minutes, and then removed for reperfusion. The day after MCAo surgery, an acute neurological assessment (in other words, forelimb and hindlimb placement test and rotational movement test shown as modified neurological severity scale [mNSS] score) was performed to select an appropriate stroke rat model. Animals with mNSS ranging from 2 points to 3 points (5: normal, 4: mild, 1: severe) were selected. In addition, before transplanting a neural precursor cell (in other words, 7 days after MCAo induction), a rat with moderate to severe sensorimotor deficits (in other words, 15 points or more on the modified neurological severity scale [mNSS] score) was finally selected for the experiment. Of the 55 rats, 12 animals were excluded for various reasons, including death before transplantation (n=7), mild neurological deficit (n=5), etc. Therefore, a total of 40 rats were used in this study.

### Experimental Example 4. Preparation of Middle Cerebral Artery Occlusion Mouse Model Administered with Human Leukocyte Antigen Homozygous Induced Pluripotent Stem Cell-Derived Neural Precursor Cells

To investigate the therapeutic effect of hiPSC-NPC, a transplantation experiment was designed in three examples as follows.
(1) Example 1 (culture medium control group, group 1): 2 μl of media (n=10)
(2) Example 2 (fibroblast control group, group 2): 2×10⁵/2 μl of fibroblasts (n=10),
(3) Example 3 (iPSC-NPC group, group 3): 2×10⁵/2 μl of hiPSC-NPCs (n=10).

Seven days after MCAo induction, the following two sites were transplanted (1×10⁵ cells/μl each).
(i) anterior-posterior (AP): +1.0 mm; medial-lateral (ML): −2.5 mm; dorsal-ventral (DV): −2 mm from the bregma,
(ii) AP: +1.0 mm; ML: −2.5 mm; DV: −7 mm from the bregma.

All animals were immunosuppressed with Cyclosporine A (15 mg/Kg: CKD Pharmaceuticals, Korea) intraperitoneal, starting from 1 day before transplantation and continuing every day throughout the study.

### Experimental Example 5. Histological Analysis

At 12 weeks after hiPSC-NPC transplantation, all animals were anesthetized by intraperitoneal injection of 1% ketamine (30 mg/kg) and xylazine hydrochloride (4 mg/kg), followed by perfusion of saline and 4% paraformaldehyde into the carotid artery. Brains were extracted and post-fixed overnight in 4% paraformaldehyde at 4° C., then transferred to 30% sucrose solution for 2 days until precipitation. Brains were frozen in OCT compound (Lot No. 3801480; Leica) and stored at −80° C. Brains were sectioned coronally at 40 μm thickness using a cryostat (Leica CM3050 S; Leica Microsystems) and stored in 24-well plates until use.

### Experimental Example 6. Confirmation of Behavioral Recovery in Middle Cerebral Artery Occlusion Mouse Model after Transplantation of Human Leukocyte Antigen Homozygous Induced Pluripotent Stem Cell-Derived Neural Precursor Cells

To determine whether hiPSC-NPC transplantation may improve behavioral abnormalities caused by middle cerebral artery occlusion (MCAo), rotarod tests, stepping test, modified neurological severity score (mNSS) test, staircase test, and apomorphine-induced rotation test were performed for 12 weeks after hiPSC-NPC transplantation, and the size of cerebral infarction was measured.

### Experimental Example 6.1. Behavioral Experiment Method

Five tests were performed to monitor behavioral changes upon hiPSC-NPC transplantation (n=10 for each group). To reduce variation between animals, rats were trained for rotarod and staircase tests three times per day under identical conditions for three consecutive days before MCAo induction. For the reference standard, rotarod, stepping, and staircase tests were performed before MCAo (pre-MCAo) and all five tests were performed 1 day after MCAo (0 W data), 1 week after MCAo (1 W data), and weekly for 12 weeks.

### Experimental Example 6.1.1. Rotarod Test

A rotarod test was performed to investigate motor function and balance control. The duration of time until the animal fell off the rotarod accelerated from 0 to 40 rpm was measured. Within a total of 120 seconds, the test was performed three times a day, weekly, and the mean time was calculated.

### Experimental Example 6.1.2. Stepping Test

A stepping test was performed to investigate sensory and motor functions. All animals were kept in the same position, and one forelimb and two hindlimbs of each animal were fixed. The unfixed forelimb of the rat was placed in contact with a board (900 mm long, for 5 seconds) and moved initially back and forth and slowly laterally by the experimenter.

The same method was used to measure the two forelimbs alternately. The number of steps the rat took with both forelimbs on the board was counted, and the average ratio of the stroke-affected and unaffected forelimbs was calculated. The test was performed three times a day, every week.

### Experimental Example 6.1.3. Modified Neurological Severity Score (mNSS) Test

A mNSS test was performed to evaluate neurological deficits in rats damaged by ischemic stroke after transplantation. The test is a composite test of motor, sensory, beam balance and reflex tests 32-35 and graded on a scale of 0-28 (normal score: 0, maximum deficit score: 28). Tests were performed weekly.

### Experimental Example 6.1.4. Staircase Test

A staircase test was performed to assess the independent use of the forelimb in a ‘site-specific’ skilled reaching and grasping tasks. Animals were pretrained before the experiment to learn how to use their forelimbs to eat a pellet placed in a concave hole. Each rat was placed on its left side in a staircase apparatus with five affected pellets for 15 minutes and the number of pellets eaten by the rat was counted. The experiment was performed once a day for three consecutive days every other week.

### Experimental Example 6.1.5. Apomorphine-Induced Rotation Test

Additionally, an apomorphine-induced rotation test was performed, which may provide a sensitive and rapid behavioral correlate of a substantia nigra. When the substantia nigra region was damaged, animals injected with apomorphine (1.0 mg/kg in saline containing 0.02% ascorbate; Sigma) were rotated toward the unaffected side. All animals were fitted with a harness with a thin steel wire that transmitted movement to an electromechanical sensor. All animals were injected intraperitoneally with apomorphine, and the number of turns was counted starting 5 minutes later and within 60 minutes. This test was performed at 0, 2, 4, 8, and 12 weeks.

### Experimental Example 6.2. Method of Measuring Infarct Size

Cresyl violet staining on 16 2-μm-thick coronal sections was performed to measure final infarct size (n=7 from each group). A total of eight serial sections from each animal were analyzed. Infarct size was defined as a percentage of the intact contralateral hemisphere using the following Equation 1.

Estimated infarct size (%)=[1−(remaining ipsilateral hemisphere area/intact contralateral hemisphere area)]×100. Equation 1

The areas of interest were measured using ImageJ software, and values were summed for eight serial coronal sections per brain.

### Experimental Example 6.3. Statistical Analysis

Statistical analysis of all experiments was performed using Prism software (version 8.0, GraphPad). Tissue analysis was performed using one-way analysis of variance (ANOVA) and behavioral performance was analyzed using two-way ANOVA. For multiple group comparisons, post hoc Tukey's b test was used. All data were presented as mean±standard error of the mean. P values<0.5 were considered statistically significant.

### Experimental Example 6.4. Results

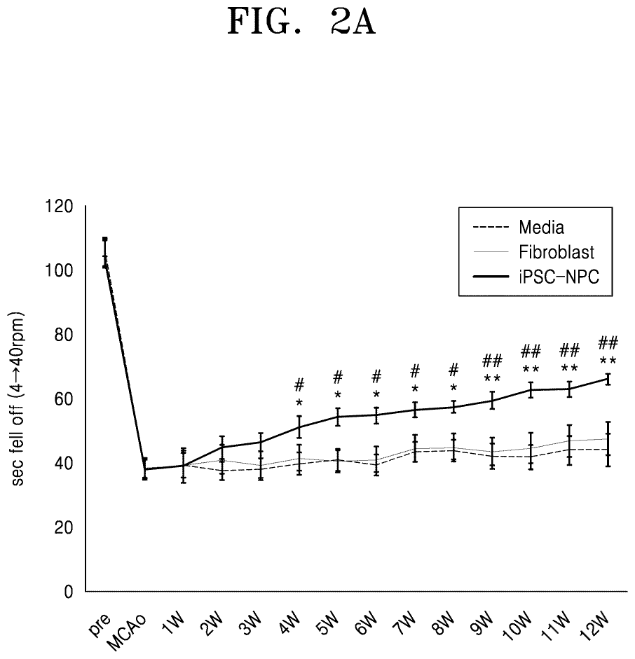

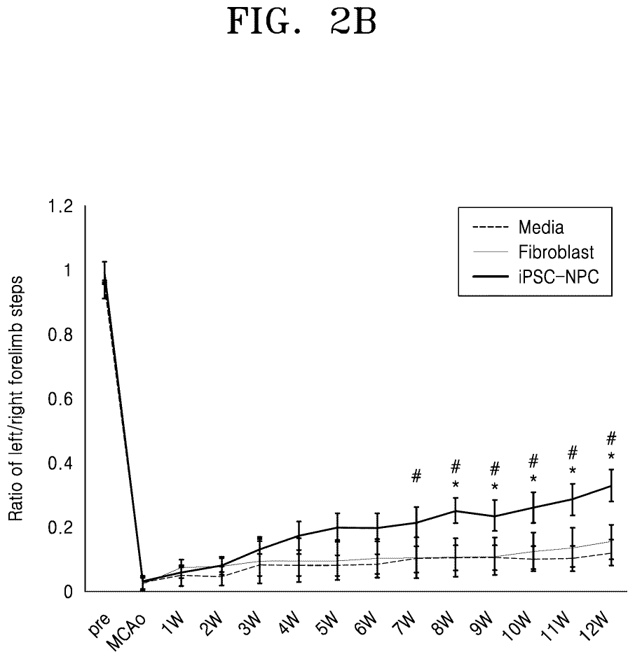

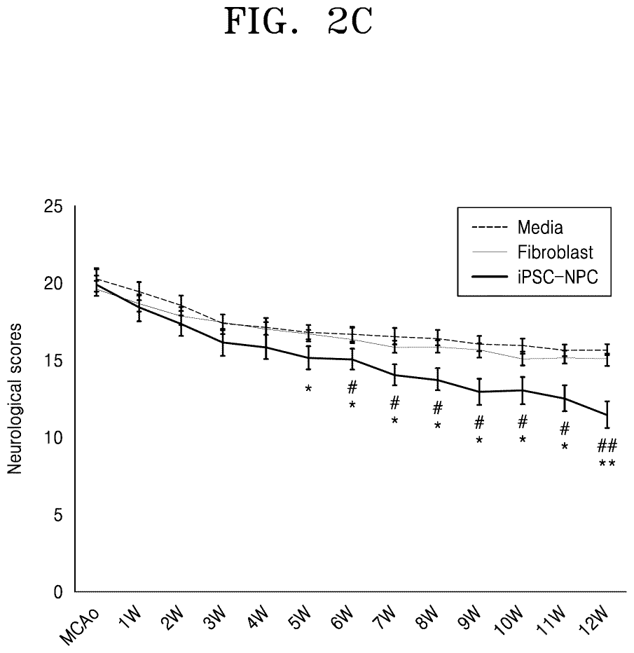

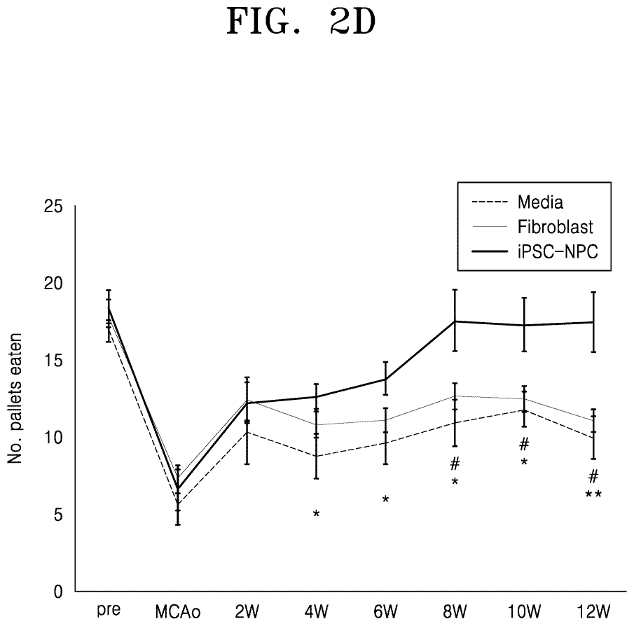

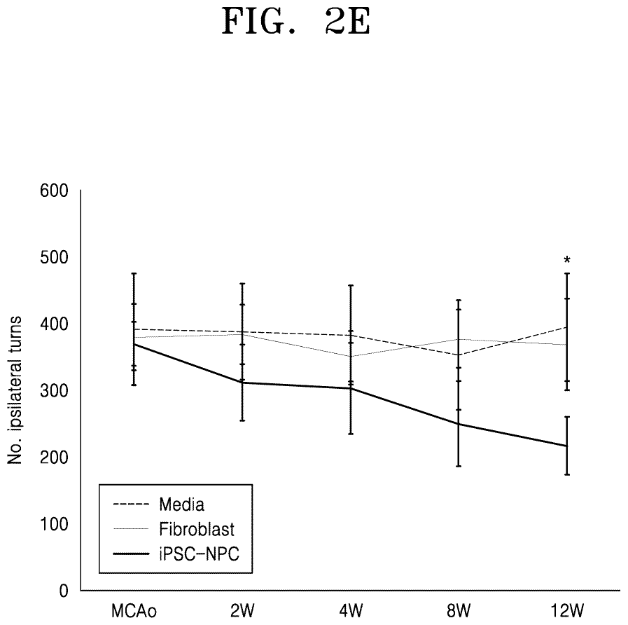

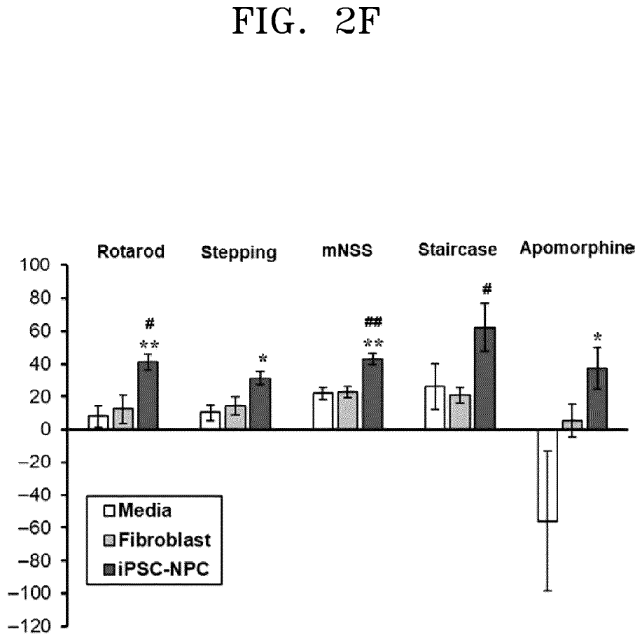

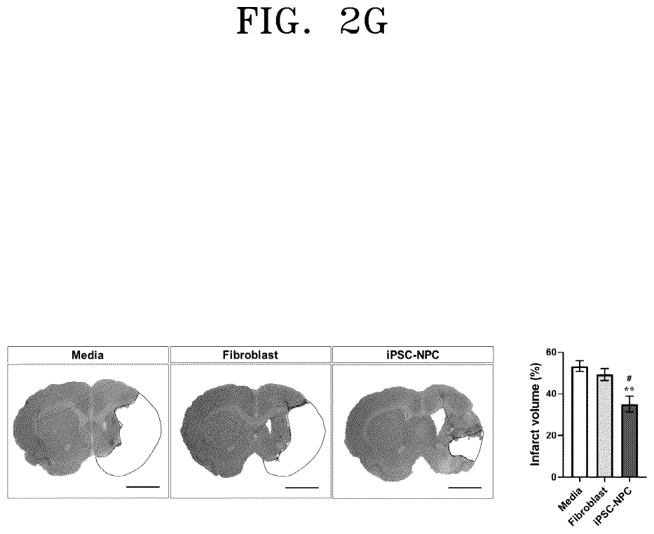

As a result, the hiPSC-NPC group showed significant improvement in all five behavioral tests compared to group transplanted only with the culture medium control group and fibroblast control group. In the rotarod test, the hiPSC-NPC group showed increased time off the bar starting at 4 weeks compared to the culture medium control group and fibroblast control group. This statistical difference was maintained up to 12 weeks (see FIG. 2A).

In the stepping test, the hiPSC-NPC group showed significant behavioral improvement from 7 weeks up to 12 weeks compared to group transplanted only with the culture medium control group and fibroblast control group (see FIG. 2B).

In the mNSS test, the hiPSC-NPC group significantly reduced neurological deficit scores from 5 weeks up to 12 weeks (see FIG. 2C).

In the staircase test, the hiPSC-NPC group showed significant behavioral improvement from 4 weeks up to 12 weeks. In addition, in the apomorphine-induced rotation test, the hiPSC-NPC group showed significant improvement compared to the two control groups at week 12 (see FIG. 2D and FIG. 2E).

The hiPSC-NPC group showed significant behavioral improvement from baseline in all five tests compared to the culture medium control group and fibroblast control group (see FIG. 2F).

In addition to behavioral improvement, the final infarct size of the hiPSC-NPC group (35.01±3.45%) was significantly reduced compared to the culture medium control group (53.35±2.47%) and fibroblast control group (49.30±2.73%) (see FIG. 2G).

These results indicate that intracerebral transplantation of hiPSC-NPCs in the subacute stage of ischemic stroke restored functional deficits in rat ischemic stroke models.

### Experimental Example 7. Confirmation of Post-Transplantation Survival and Engraftment of Human Leukocyte Antigen Homozygous Induced Pluripotent Stem Cell-Derived Neural Precursor Cells

To determine whether hiPSC-NPCs may survive and engraft after transplantation, immunohistochemical experiments for various cell markers were performed.

### Experimental Example 7.1. Immunohistochemical Experiment Method

An immunohistochemical experiment was performed in the following method. A free-floating brain section was washed three times for 15 minutes in PBS, three times for 10 minutes in tPBS solution including 0.3% Triton X-100 (Sigma, USA), and then blocked in tPBS solution including 5% normal horse serum for 60 minutes at room temperature. The tissue section was incubated with the primary antibody for 12 hours at 4° C. (see Table 2).

TABLE 2

| Primary | | Host | | Catalogue |
|---|---|---|---|---|
| antibodies | Dilution | species | Suppliers | number |
| Human nuclei | 1:100 | mouse | Millipore | MAB1281 |
| (hNu) | | | | |
| Human | 1:100 | rabbit | Millipore | AB3598 |
| mitochondria | | | | |
| (hmito) | | | | |
| STEM121 | 1:250 | mouse | Takara | Y40410 |
| hNestin | 1:200 | rabbit | R&D System | 841901 |
| hNestin | 1:250 | mouse | Abcam | ab22035 |
| Ki67 | 1:200 | mouse | Invitrogen | 14-5699-82 |
| GABA | 1:250 | rabbit | Sigma-Aldrich | A2052 |
| DARPP-32 | 1:250 | rabbit | Cell Signaling | MAB9407621 |
| TH | 1:250 | rabbit | Pel-Freez | P40101-150 |
| hMap2 | 1:250 | rabbit | Santa Cruz | SC-20172 |
| NeuN | 1:250 | mouse | Millipore | MAB377 |
| GFAP | 1:500 | mouse | BD Biosciences | MAB2594 |
| GFAP | 1:250 | rabbit | Dako | Z033429 |
| Iba1 | 1:250 | rabbit | Wako | 019-19741 |
| ED1 | 1:250 | mouse | Serotec | MCA341R |
| CD206 | 1:200 | goat | Santa Cruz | SC-34577 |
| iNOS | 1:200 | rabbit | Santa Cruz | SC-649 |
| BrdU | 1:200 | mouse | BD Biosciences | 555627 |
| DCX | 1:250 | rabbit | Cell Signaling | 4604S |
| RECA1 | 1:250 | mouse | Abcam | Ab9774 |
| PECAM1 | 1:200 | goat | R&D Systems | AF3628 |
| vWF | 1:200 | rabbit | Invitrogen | PA5-104687 |

Afterwards, the tissue section was washed five times for 10 minutes in PBS and then reacted in the corresponding fluorescence-conjugated secondary antibody against each primary antibody for 90 minutes. The tissue section as then washed in PBS for 10 minutes and then incubated in DAPI (1:500, Roche, USA) stain for 30 minutes to label the cell nuclei. The secondary antibodies used in this study were as follows:

Goat anti-mouse IgG-conjugated Alexa-488 (1:250; Invitrogen), goat anti-rabbit IgG-conjugated Alexa-488 (1:250; Invitrogen), goat anti-mouse IgG-conjugated Alexa-555 (1:250; Invitrogen), goat anti-rabbit IgG-conjugated Alexa-555 (1:250; Invitrogen) and donkey anti-goat IgG-conjugated Alexa-555 (1:250; Invitrogen).

### Experimental Example 7.2. 5′-Bromo-2′-deoxyuridine (BrdU) Injection

After cell transplantation, 0.5 μl of 4% Fluoro-Gold (FG) (Fluorochrome) was stereotactically injected into the ipsilateral globus pallidus (AP: −1.3 mm, ML: −3.4 mm, DV: −6.5 mm) of MCAo at 12 weeks.

Additionally, to detect endogenously proliferating stem cells, 5′-bromo-2′-deoxyuridine (BrdU) (50 mg/kg; Sigma-Aldrich) was injected intraperitoneally for 1 week at 12-hour intervals prior to sacrifice (n=3 for each group).

Fluorescently labeled tissue sections were imaged using a confocal laser scanning microscope (Leica TCS SP5 II, Leica Microsystems, Germany). BrdU-positive cells were detected by immunohistochemical method using an antibody against BrdU after denaturation of DNA in 1 M HCl for 30 minutes at 45° C. Secondary antibody incubation, counter staining and confocal analysis procedures were identical to those described above.

### Experimental Example 7.3. Cell Counting

For cell count measurements, three coronal sections cut at 40 μm thickness (AP: +1.0, 0, and −1.0 mm) from each animal were used after double immunohistochemical staining. Stereoscopic quantification of a co-labelled cell was performed in a region of interest (ROI) of the cortex and striatum of the ischemic penumbra and boundary regions under a 40× objective from a confocal laser-scanning microscope. To examine the survival and differentiation of transplanted human cells, 13 brain tissue sections starting from AP: +1.5 mm to AP: −1.5 mm in each animal were analyzed using the 40× objective of a confocal laser-scanning microscope. Data were expressed as percentage of positive cells.

To investigate changes in inflammatory responses in the host brain following transplantation, 5 ROIs were analyzed in the ischemic boundary region using a 40× objective. Data were presented as the percentage of positive cells out of total DAPI-positive cells. To measure changes in glial scar formation, five ROIs adjacent to the ischemic area of the GFAP-positive area were quantified. The area of glial scar formation and its thickness were measured as described above. Data were presented as mean area (μm²)/ROI and mean thickness (μm)/ROI.

To examine changes of endogenous neurogenesis, proliferating cells were counted in three regions of the subventricular zone (SVZ). For this purpose, the number of BrdU+ cells alone, DCX+ cells alone, and BrdU+-DCX+co-labelled cells were counted at 5 ROIs within the ipsilateral SVZ wall and data were presented as percentage of positive cells out of DAPI-positive cells.

For quantitative measurements of cerebral blood vessels, RECA1⁺ blood vessels formed by endothelial cells were counted at 4 ROIs in the ischemic penumbra under a 10× objective of an optical microscope (Nikon Eclipse E600) (n=5 in each group). All cell counting analyses were performed using ImageJ software (NIH).

### Experimental Example 7.4. Results

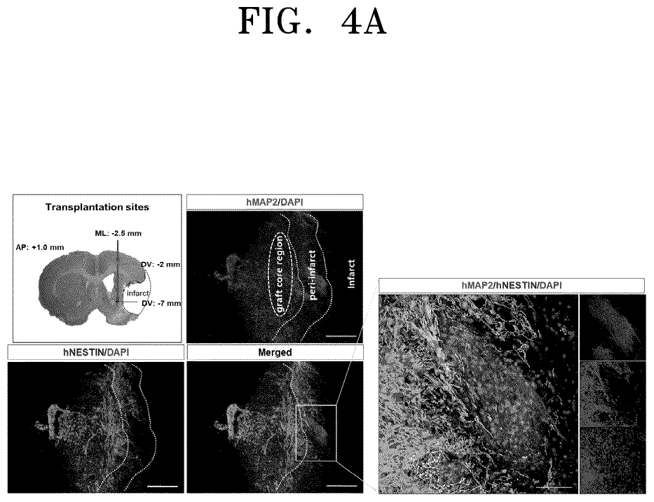

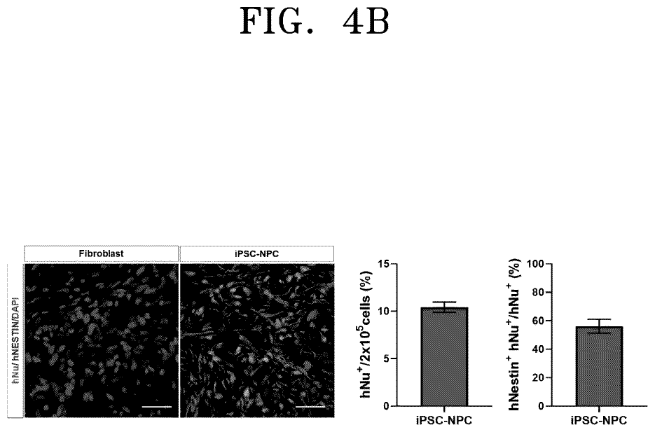

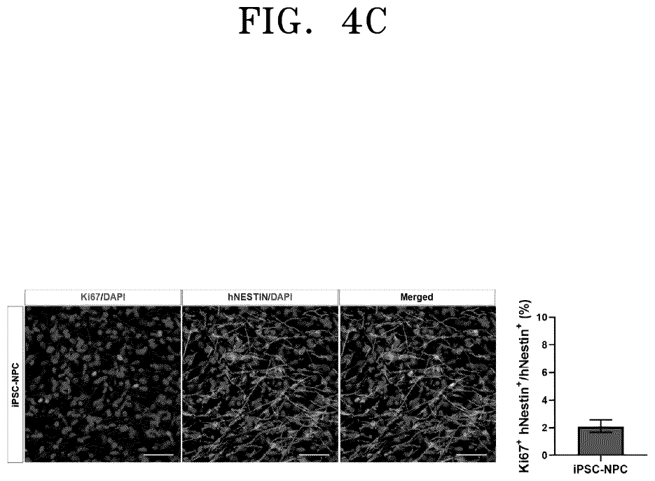

Precise quantitative analysis of hNestin⁺ cells was not easy due to the high density of the cell bodies and the protruding terminal regions. However, it was observed that at 12 weeks after transplantation, a significant portion of the transplanted cells were engrafted at the injection site in the iPSC-NPC group (see FIG. 3A and FIG. 3B). The majority of transplanted cells in the core region were undifferentiated Nestin⁺ NPCs (see FIG. 4A and FIG. 4B (left)). However, a significant portion of transplanted cells outside the graft core towards the infarct region differentiated into hMAP2⁺ mature neurons. Although no transplanted cells were detected in the fibroblast group, hNut and hNestin⁺ cells clearly engrafted at 12 weeks after transplantation.

Quantitative analysis of DAB immunostaining for hNu detected a total of 20,846±1,087 hNu⁺ cells, which accounted for 10.42±0.54% of the transplanted cells (see FIG. 4B (center)).

Approximately 56.10±5.01% of the transplanted cells merged with hNu and hNestin, indicating that a significant portion of the transplanted cells still remained as neural precursor cells at 12 weeks after transplantation (see FIG. 4B (right)).

Only 2.12±0.45% were observed to be positive for Ki67, a marker for proliferating cells, confirming that most of engrafted NPCs remained in a non-proliferating state (see FIG. 4C).

This means that most of human leukocyte antigen homozygous induced pluripotent stem cell-derived neural precursor cells survive and engraft after transplantation.

### Experimental Example 8. Confirmation of Post-Transplantation Effects of Human Leukocyte Antigen Homozygous Induced Pluripotent Stem Cell-Derived Neural Precursor Cells

### Experimental Example 8.1. Confirmation of Differentiation into Neurons and Glial Cells

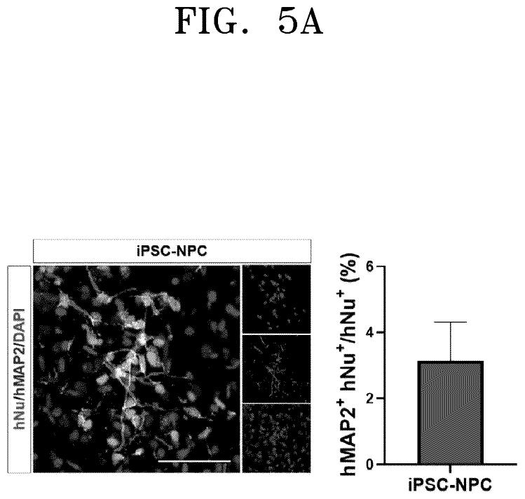

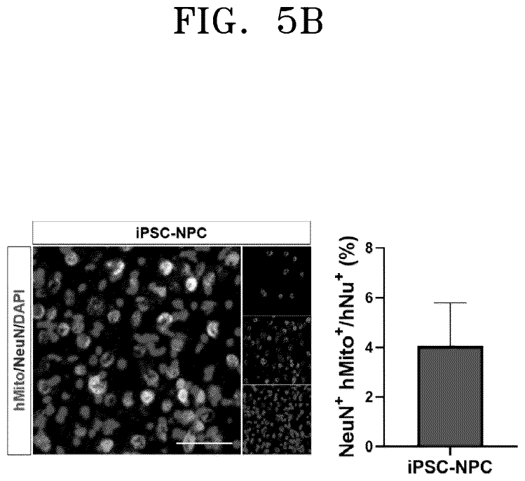

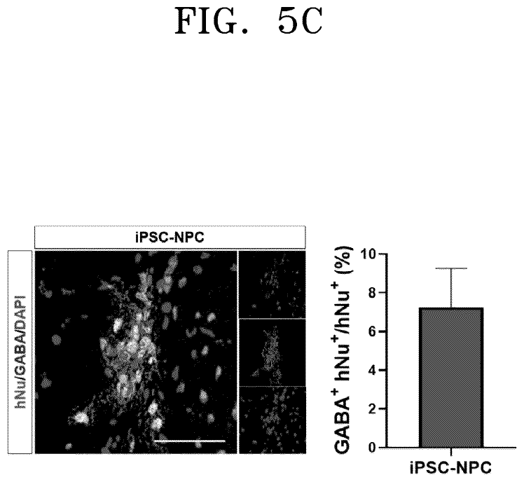

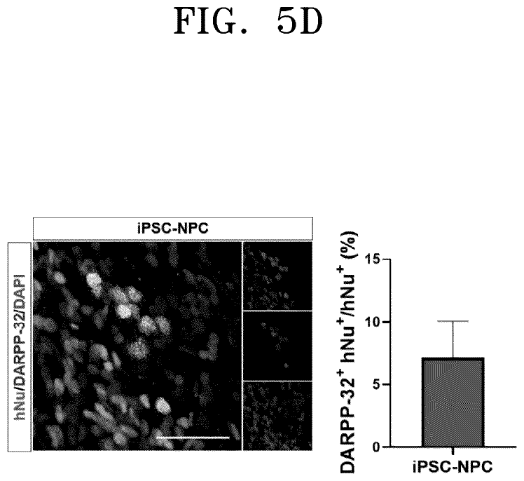

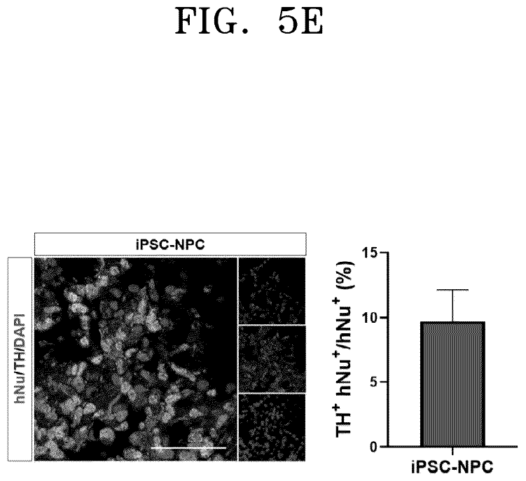

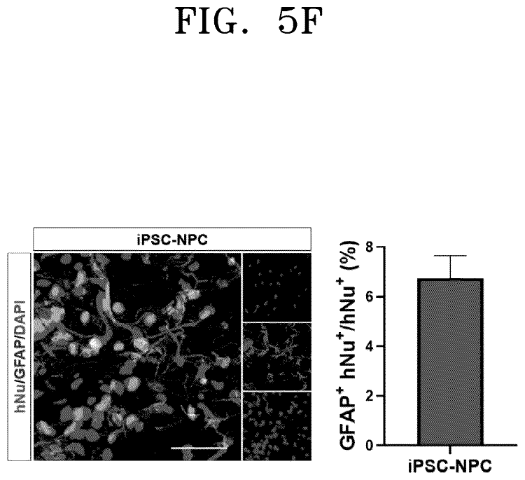

To determine whether the transplanted neural precursor cells were a more differentiated form and potentially able to induce neural replacement, a quantitative analysis of the neuronal and glial differentiation from transplanted neural precursor cells in the peri-infarct area (see FIG. 5A).

As a result, approximately 1,329±846 hNu⁺ cells in the hiPSC-NPC group were detected in the peri-infarct area, where they were differentiated into hMAP2⁺ or NeuN⁺ mature neurons (see FIG. 5A and FIG. 5B).

It was confirmed that the transplanted cells differentiated into GABAergic neurons (see FIG. 5C), DARPP-32⁺ medium spiny neurons (see FIG. 5D) or TH⁺ dopaminergic neurons (see FIG. 5E), and GFAP⁺ astroglial cells (see FIG. 5F).

It suggests that human leukocyte antigen homozygous induced pluripotent stem cell-derived neural precursor cells were able to differentiate into neurons and glial cells after transplantation and replace existing nerves.

### Experimental Example 8.2. Confirmation of Neural Connections

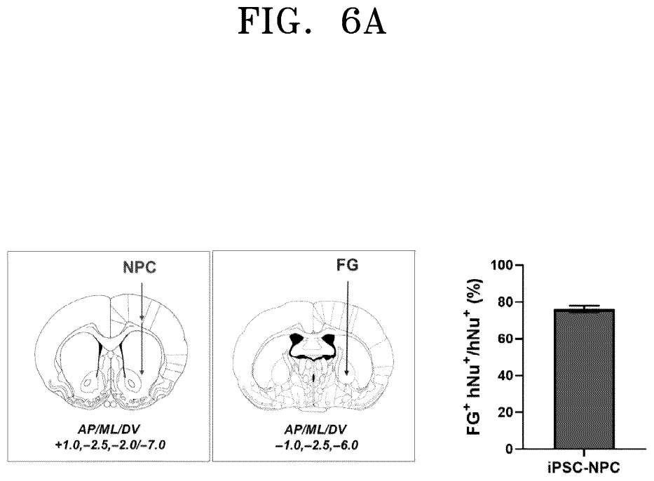

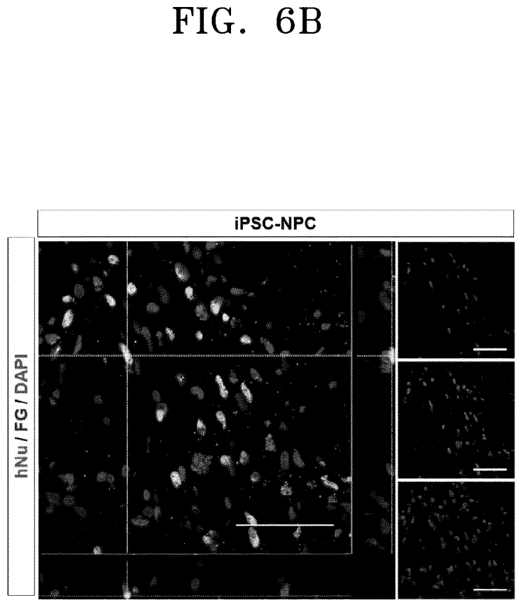

To investigate whether the transplanted cells can form neural connection with host brain cells, a retrograde neurotracer, Fluoro-Gold (FG), was injected into the ipsilateral globus pallidus, and the co-labeled hNu+-FG+ cells were analyzed in the ipsilateral striatum (see FIG. 6A (left)).

As a result, a significantly high proportion (76.22±1.80%) of the transplanted hiPSC-NPCs were positive for FG signaling (see FIG. 6A (right) and FIG. 6B).

It suggests that the transplanted neural precursor cells were successfully connected with host striatal neurons, forming a neuronal network between the transplanted cells and the host brain.

### Experimental Example 8.3. Confirmation of Host Immune Responses and Gliosis

To determine changes in host immune responses and gliosis after transplantation, microglial activation, proportion of different microglial phenotypes, and degree of glial scar formation were determined.

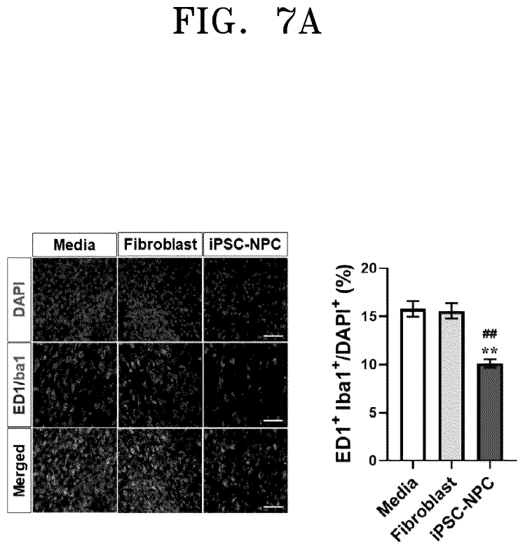

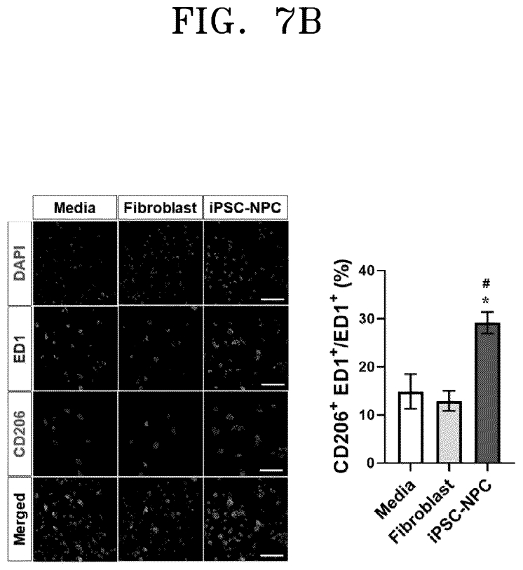

As a result, in the brains of MCAo mice, numerous microglia (Iba1⁺ cells) were found in the peri-infarct area, some of which were in an activated form (ED1⁺ cells). It was observed that ED1⁺-Iba1⁺ cells, which are activated phagocytes, were significantly reduced in the iPSC-NPC group compared with the levels of the culture medium control group and fibroblast control group (see FIG. 7A).

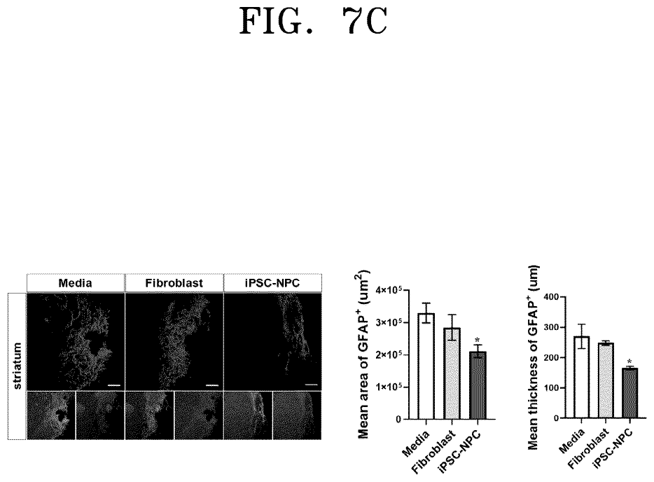

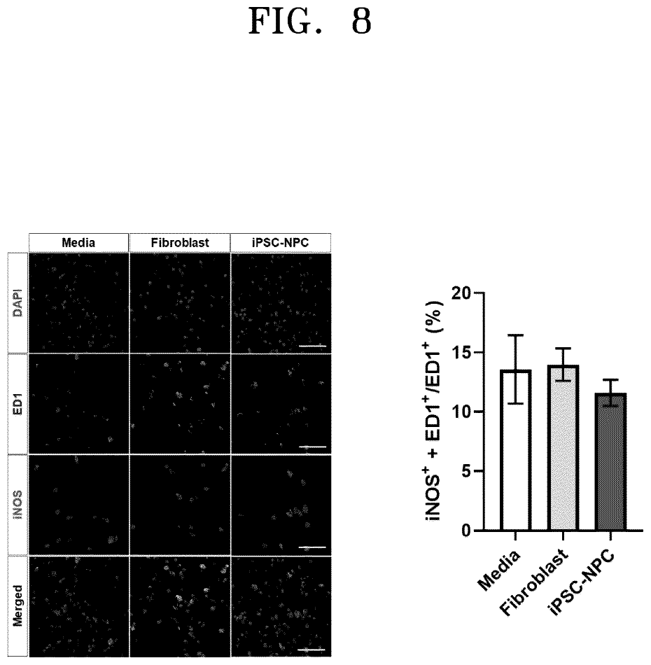

In addition, double immunostaining was performed on iNOS⁺-ED1⁺ cells and CD206⁺-ED1⁺ cells to further investigate the proportion of different microglial phenotypes. As a result, the proportion of CD206⁺-ED1⁺ cells increased significantly in the iPSC-NPC group compared with the levels of the culture medium control group and the fibroblast control group (see FIG. 7B). The proportion of iNOS⁺-ED1⁺ cells was not significantly different among the three groups (see FIG. 8).

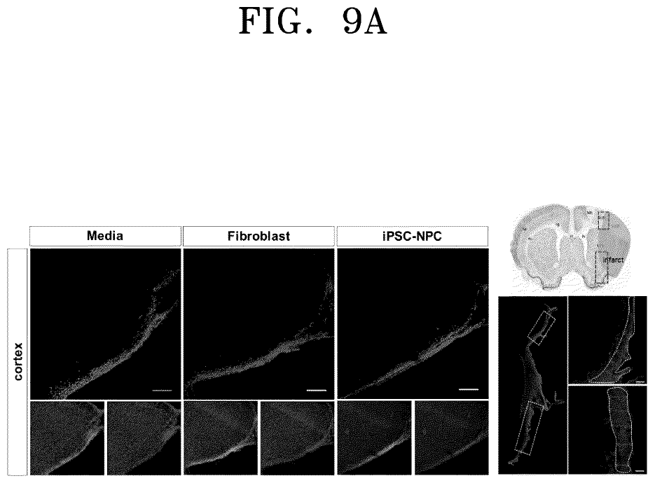

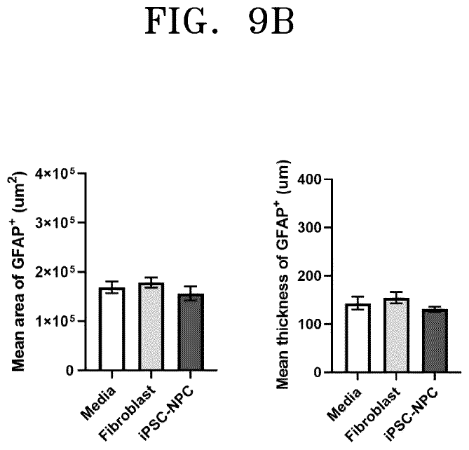

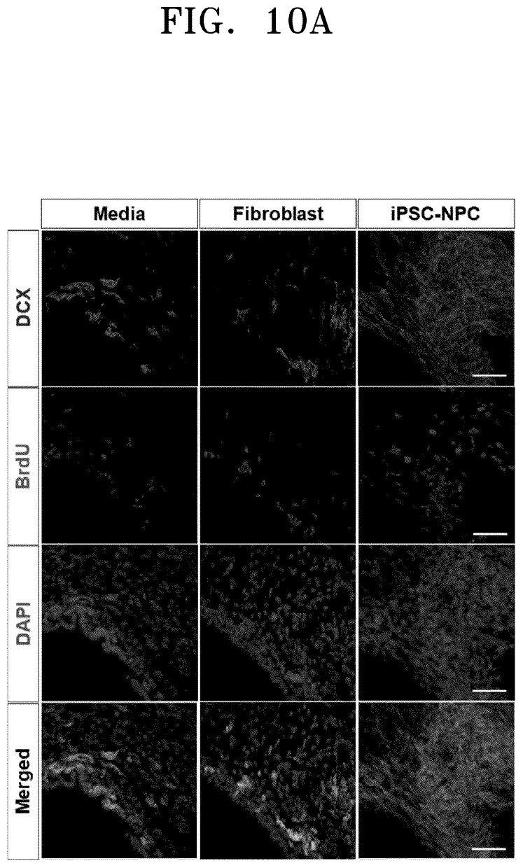

Next, immunostaining was performed using an antibody against GFAP to assess the glial scar area and the thickness of the ipsilateral hemisphere (see FIG. 7C (left)). No difference was observed in the area and thickness of GFAP+ glial scars among the three groups in the peri-infarct cortex (see FIG. 9A and FIG. 9B).

However, in the peri-infarct striatal region, the area of glial scars in the hiPSC-NPC group was reduced compared with that in the culture medium control group and fibroblast control group, and the thickness of glial scars was also reduced in the hiPSC-NPC group compared with the levels in the culture medium control group and fibroblast control group (See FIG. 7C).

These results suggest that transplantation of hiPSC-NPC not only reduces post-stroke neuroinflammation but also promotes the healing process of damaged brain and prevents glial scar formation in the subacute phase of ischemic stroke in rats.

### Experimental Example 8.4. Confirmation of Endogenous Neurogenesis

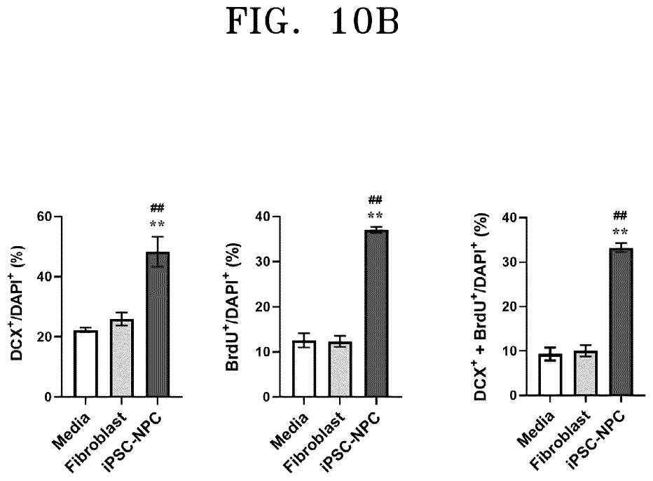

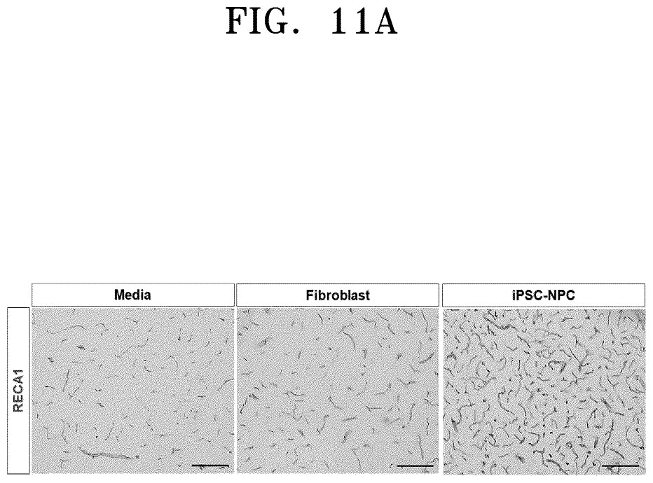

In ischemic brain injury, endogenous neural precursor cells generated in the subventricular zone (SVZ) are known to migrate towards the injury site to replace the lost brain cells. To investigate whether transplanted neural precursor cells can affect endogenous SVZ neurogenesis, double staining for BrdU and DCX was performed (see FIG. 10A).

As a result, immunohistochemical analysis revealed that the number of DCX⁺ neuroblasts was significantly increased in the hiPSC-NPC group compared with the levels in the culture medium control group and fibroblast control group (see FIG. 10B (left)). The number of BrdU+ proliferating cells in the ipsilateral SVZ was significantly increased in the hiPSC-NPC group compared with levels in the culture medium control group and fibroblast control group (see FIG. 10B (center)). The number of BrdU and DCX double positive cells (in other words, proliferating neuroblasts) was significantly increased in the hiPSC-NPC group compared with the levels in the culture medium control group and fibroblast control group (see FIG. 10B (right)).

This means that transplantation of hiPSC-NPCs enhances SVZ neurogenesis in the damaged brain after stroke.

### Example 8.5 Confirmation of Cerebral Angiogenesis Development

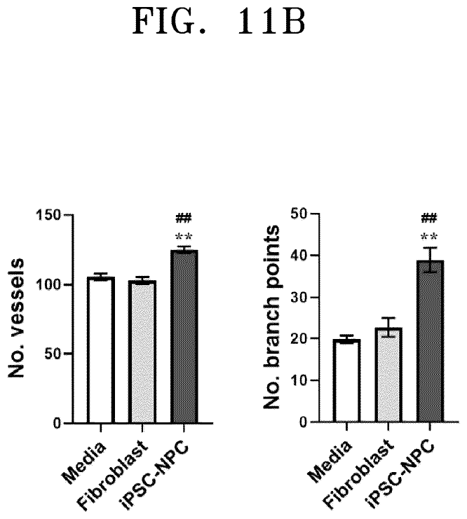

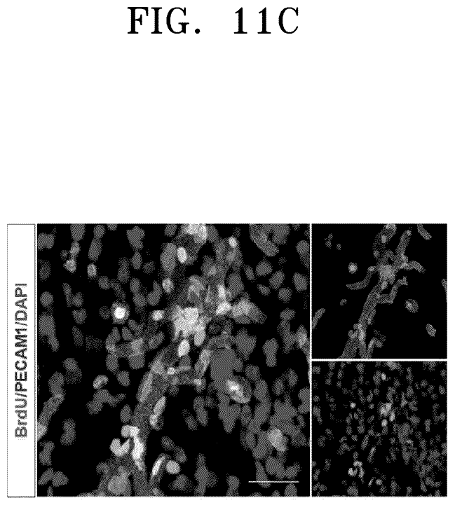

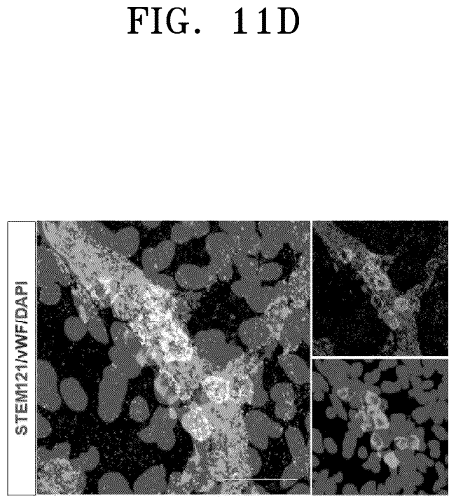

After transplantation of neural precursor cells, immunohistochemical analysis was performed to determine whether new blood vessel formation may be induced in the perilesional area (see FIG. 11A).

For the quantitative measurements of cerebral blood vessels, RECA1⁺ blood vessels formed by endothelial cells were counted in 4 ROIs of the ischemic penumbra under a 10× objective lens of an optical microscope (Nikon eclipse E600) (n=5 in each group). All cell counting analyses were performed using ImageJ software (NIH, USA).

As a result, it was confirmed that the number of RECA1⁺ blood vessels (diameter less than 30 μm) in the peri-infarct area was significantly increased in the hiPSC-NPC group compared with the levels in the culture medium control group and fibroblast control group (see FIG. 11B (left)). The number of capillary bifurcations was also significantly increased in the hiPSC-NPC group compared with those in the culture medium control group and fibroblast control group. In addition, it was confirmed that BrdU⁺ proliferating cells were present around the PECA1⁺ vessels (see FIG. 11B (right) and FIG. 11C). It was confirmed that the transplanted hiPSC-NPC, labeled as STEM121+ cells, were presented around RECA1+ vascular endothelial cells in the peri-infarct area (see FIG. 11D).

These results suggest that the transplanted hiPSC-NPCs are actively involved in the repair process of peri-infarct blood vessels and promote angiogenesis following stroke in rats.

1. A pharmaceutical composition for preventing or treating a stroke, the pharmaceutical composition comprising:
a neural precursor cell differentiated from an HLA homozygous human induced pluripotent stem cell (hiPSC-NPC) comprising an HLA homozygote as an active ingredient.

2. The pharmaceutical composition of claim 1, wherein the HLA homozygous is homozygous for HLA-A, HLA-B, and HLA-DRB1.

3. The pharmaceutical composition of claim 1, wherein the human induced pluripotent stem cell is derived from a cord blood mononuclear cell (CMC) or a peripheral blood mononuclear cell (PBMC).

4. The pharmaceutical composition of claim 1, wherein the neural progenitor cell comprises a gene selected from the group consisting of Sox2, Nestin, and Musashi.

5. The pharmaceutical composition of claim 1, wherein the stroke is selected from the group consisting of a cerebral infarction, a cerebral hemorrhage, a transient ischemic attack, and a recurrent stroke.

6. The pharmaceutical composition of claim 1, wherein prevention or treatment of the stroke is achieved by cerebral angiogenesis.

7. A method of producing a neural precursor cell differentiated from a HLA homozygous human induced pluripotent stem cell comprising an HLA homozygote, the method comprising:
differentiating the HLA homozygous human induced pluripotent stem cell comprising the HLA homozygote into the neural precursor cell.

8. The method of claim 7, wherein a differentiation process is performed in a differentiation medium for neural precursor cell differentiation.

9. The method of claim 8, wherein the differentiation medium is coated with poly-L-ornithine or laminin.

10. The method of claim 7, further comprising:
forming an embryoid body (EB) containing the neural precursor cell.
